(19) United States
(12) Patent Application Publication
(10) Pub. No.: US 2026/0249276 A1
(43) Pub. Date: Aug. 27, 2026

(54) DENDRITIC MIXED METAL OXIDE CATALYST AND METHOD OF USING THE SAME FOR CO2 HYDROGENATION

(71) Applicant: Saudi Arabian Oil Company, Dhahran (SA)

(72) Inventors: Mohnnad H. Alabsi, Thuwal (SA); Ali Harbi, Thuwal (SA)

(21) Appl. No.: 19/061,566

(22) Filed: Feb. 24, 2025

(51) Int. Cl. B01J 23/83; B01J 23/10; B01J 35/30; B01J 35/40; B01J 35/45; B01J 35/51; B01J 35/61; B01J 35/63; B01J 35/64; B01J 37/00; B01J 37/02; B01J 37/08; C07C 1/12

(52) U.S. Cl. CPC B01J 23/83; B01J 23/10; B01J 35/394; B01J 35/40; B01J 35/45; B01J 35/51; B01J 35/613; B01J 35/615; B01J 35/633; B01J 35/635; B01J 35/647; B01J 35/651; B01J 37/0018; B01J 37/0201; B01J 37/08; C07C 1/12

(57) ABSTRACT

The present disclosure provides a composition. The composition includes a catalyst. The catalyst includes a mixed metal oxide support having a dendritic geometry and a metal coupled the metal oxide support.

### TECHNICAL FIELD

Embodiments of the present disclosure generally relate to carbon dioxide hydrogenation. More specifically, embodiments of the present disclosure relate to a dendritic mixed metal oxide catalyst and method of using the same for carbon dioxide hydrogenation.

### BACKGROUND

There is a global endeavor to develop technology aimed at mitigating the effects of climate change. The rise in carbon emissions has garnered the attention and apprehension of several nations and global organizations. The concentration of carbon dioxide (CO₂) in the Earth's atmosphere has experienced an increase of 45 parts per million (ppm) over the last two decades. This growth is regarded as a matter of concern due to its potential association with several environmental phenomena, including desertification, sea level rise, and an overall elevation in global average temperatures. Some countries have established specific objectives to decrease their carbon dioxide emissions. As an illustration, the Kingdom of Saudi Arabia hopes to decrease its carbon emissions by 278 million metric tons per annum (MTPA) by the year 2030. Furthermore, the nation hopes to achieve a state of net zero emissions by the year 2060.

Transportation fuels make a substantial contribution to the release of carbon dioxide into the atmosphere Consequently, the process of decarbonizing fuels holds considerable potential in mitigating CO₂ emissions. As an example, the transformation of CO₂ into gasoline exhibits the capacity to mitigate the emission of this particular greenhouse gas by a substantial amount, for example, it is conceived by up to 6000 million metric tons (MMT).

### SUMMARY

An embodiment of the present disclosure relates to a composition. The composition includes a catalyst including a mixed metal oxide support having a dendritic geometry and a metal coupled the metal oxide support. In some embodiments, when the catalyst contacts carbon dioxide and hydrogen, the catalyst produces a product stream comprising an unsubstituted straight or branched chained C₅₋₁₅ alkane.

In another embodiment, the present disclosure provide a reactor that includes: an inlet in fluid communication with a carbon dioxide source and a hydrogen source, the carbon dioxide source comprising carbon dioxide, and the hydrogen source comprising hydrogen; and a catalyst as described in the preceding paragraph. The catalyst is in the reactor.

In a further embodiment, the present disclosure provides a method. The method includes contacting a catalyst with carbon dioxide and hydrogen to produce a product stream a product stream comprising an unsubstituted straight or branched chained C₅₋₁₅ alkane. In some embodiments, the catalyst includes a mixed metal oxide support having a dendritic geometry and a metal coupled the metal oxide support.

In yet another embodiment, the present disclosure provides a method. The method includes adding a surfactant and a templating agent to a first solvent to produce a first solution and adding a silica precursor to the first solution to produce a silica support having a dendritic geometry. The method includes calcining the silica support having the dendritic geometry and impregnating the silica support having the dendritic geometry with a first metal salt and a second metal salt. The method includes calcining the silica support having the dendritic geometry with the first metal salt and the second metal salt to produce a dried product comprising a mixed metal oxide coupled to the silica support having the dendritic geometry and contacting the dried powder with an etching agent, wherein the etching agent removes the silica support to generate a mixed metal oxide support having the dendritic geometry.

The details of one or more implementations of the subject matter of this specification are set forth in the Detailed Description, the accompanying drawings, and the claims. Other features, aspects, and advantages of the subject matter will become apparent from the Detailed Description, the claims, and the accompanying drawings.

### DESCRIPTION OF THE DRAWINGS

FIG. 1 is a schematic drawing of a reactor according to an embodiment of the present disclosure.

FIG. 2 is a schematic flowchart illustrating a method of hydrogenating carbon dioxide according to an embodiment of the present disclosure.

FIG. 3 is a schematic flowchart illustrating a method of synthesizing a catalyst according to an embodiment of the present disclosure.

FIG. 4 is graph of X-ray diffraction (XRD) patterns of catalysts according to an embodiment of the present disclosure.

FIG. 5 is a graph of selectivity of catalysts that hydrogenated CO₂ at a temperature of 350° C., a pressure of 50 bar, and at a gas hourly space velocity (GHSV) of 3,300.

Like reference numbers and designations in the various drawings indicate like elements.

### DETAILED DESCRIPTION

The composition and method described herein are directed to a catalyst and a method of using the same to hydrogenate carbon dioxide into a product stream that comprises fuel range products, octane boosters, or a combination thereof (e.g., C₅₊ products, olefins, alcohols, or a combination thereof). The provided composition and method provide practical applications and technical advantages that address various technical issues associated with hydrogenating carbon dioxide to fuel range and octane booster products.

There are two main approaches for hydrogenating CO₂. The first approach for hydrogenating CO₂ is via a reverse water-gas shift (RWGS) process, which includes a first hydrogenation step to produce carbon monoxide (CO) (e.g., RWGS:CO₂+H₂↔CO+H₂O), followed by a Fischer-Tropsch (FT) reaction, or otherwise oligomerization, cracking, or aromatization reactions to produce the desired fuel. The second approach for hydrogenating CO₂ is by a methanol synthesis process (e.g., CO₂+3H₂↔CH₃OH+H₂O), which is followed by a methanol-to-hydrocarbon reaction using a zeolite. A first technical issue associated with these two approaches is the high selectivity for undesired carbon monoxide (CO). For example, CO may be more than half of the total products when the second approach based on methanol is followed. Further, zeolite-based supports have drawbacks in that zeolites limit diffusion and mass transfer of reactants. A second technical issue associated with these two approaches is that each typically involves cascade reactions (e.g., at least two separate reactions with different catalysts). That is, neither approach directly converts CO₂ into fuel range products in a single reaction or reactor.

Embodiments of the present disclosure include a catalyst that can provide practical applications and technical features that address the aforementioned potential issues. In some embodiments, the catalyst includes a mixed metal oxide support having a dendritic geometry and a metal coupled to the metal oxide support. When the catalyst contacts carbon dioxide and hydrogen, the catalyst produces a product stream comprising fuel range products (e.g., an unsubstituted straight or branched chain C₅₊ alkane, an unsubstituted straight or branched chain C₂₊ olefin, an unsubstituted straight or branched chain C₂₊ alcohols, or a combination thereof). Unlike the two above-mentioned approaches, in some embodiments, the provided catalyst may directly hydrogenate CO₂ into the fuel range products. For example, the provided catalyst may directly hydrogenate CO₂ into the fuel range products in a single reaction or reactor operating at a reaction condition (e.g., temperature, pressure, etc.), whereas the two above-mentioned approaches typically involve multiple cascade reactions that utilize different catalysts for each reaction, and which operate at different reaction conditions during the cascade reactions (e.g., different temperatures, different pressures, etc.). By allowing for the direct hydrogenation of CO₂, the catalyst of the present disclosure can provide a first technical improvement that can lower cost associated with the hydrogenation process.

Additionally, in some embodiments, the provided catalyst provides a second technical improvement by reducing the production of undesired byproducts, such as CO. In some embodiments, the provided catalyst hydrogenates CO₂ to produce the product stream with a CO selectivity that is from 0% to 1% (e.g., less than 1%, less than 0.5%, less than 0.1%, or 0%). As used herein, “selectivity” expressed as a percentage may refer to (moles of CO₂ converted to a specified product)/(moles of CO₂ consumed)*(100)−(selectivity, %).

Further, in some embodiments, the provided catalyst provides a third technical improvement by obtaining a high CO₂ conversion activity. For example, the provided catalyst may have a CO₂ conversion of at least 25% (e.g., at least 26%, at least 27%, at least 28%, at least 29%, at least 30%) and/or to at most 40% (e.g., at most 39%, at most 38%, at most 37%, at most 36%, at most 35%, at most 34%, at most 33%, at most 32%, at most 31%). As used herein, “conversion” expressed as a percentage may refer to (moles of CO₂ reacted)/(moles of CO₂ fed)*(100)−(conversion, %).

In some embodiments, the catalyst includes a mixed metal oxide having a dendritic geometry. In some embodiments, the dendritic geometry includes a central core having a plurality of arms that extend from the central core, where the arms branch at a plurality of branching points to form a spherical geometry (e.g., nanoparticle). For example, the arms may include root arms (e.g., root dendrites), terminal arms (e.g., terminal dendrites), and intermediate arms (e.g., intermediate dendrites). In some embodiments, the root arms are directly coupled to the central core. In some embodiments, the terminal arms are positioned at the outer shell of the spherical geometry, and intermediate arms couple the root arms to the terminal arms. The intermediate arms may branch a plurality of times at branching points positioned between the terminal arms and the root arms. The branching of the arms in the dendritic geometry may form a plurality of interconnected pores that are dispersed throughout the dendritic geometry of the mixed metal oxide.

In some embodiments, the catalyst is mesoporous. In some embodiments, the catalyst includes pores having a pore diameter that range from at least 2 nanometers (nm) to at most 50 nm, from at least 2 nm to at most 30 nm, from at least 2 nm to 10 nm, or from at least 2 nm to at most 7 nm. In some embodiments, the catalyst includes pores having a pore diameter of at least 2 nm (e.g., at least 3 nm, at least 4 nm, at least 5 nm) and/or to at most 50 nm (e.g., at most 40 nm, at most 35 nm, at most 30 nm, at most 25 nm, at most 20 nm, at most 15 nm, at most 10 nm, at most 9 nm, at most 8 nm, at most 7 nm, at most 6 nm). In some embodiments, the pore diameter may be calculated using any suitable method, such as gas adsorption analysis (e.g., a Barrett-Joyner-Halenda method), mercury intrusion porosimetry, scanning electron microscopy, or transmission electron microscopy.

In some embodiments, the catalyst has a pore volume from at least 0.1 cubic centimeters per gram (cc/g) to at most 0.8 cc/g. In some embodiments, the catalyst have a pore volume from at least 0.1 cc/g (e.g., at least 0.2 cc/g, at least 0.25 cc/g, at least 0.3 cc/g, or at least 0.35 cc/g) to at most 0.8 cc/g (e.g., at most 0.75 cc/g, at most 0.7 cc/g, at most 0.65 cc/g, at most 0.6 cc/g, at most 0.55 cc/g, at most 0.5 cc/g, at most 0.4 cc/g, or at most 0.35 cc/g). In some embodiments, the pore volume may be calculated using any suitable method, such as gas adsorption analysis (e.g., a Barrett-Joyner-Halenda method or Brunauer-Emmett-Teller method), mercury intrusion porosimetry, scanning electron microscopy, or transmission electron microscopy.

In some embodiments, the catalyst has a surface area from at least 40 square meters per gram (m²/g) to at most 200 m²/g. In some embodiments, the catalyst has a surface area of at least 40 m²/g (e.g., at least 50 m²/g, at least 60 m²/g, at least 70 m²/g, at least 80 m²/g) and/or to at most 200 m²/g (e.g., at most 150 m²/g, at most 125 m²/g, at most 110 m²/g, at most 100 m²/g, at most 95 m²/g, at most 90 m²/g, at most 85 m²/g). In some embodiments, the surface area may be calculated using any suitable method, such as gas adsorption analysis (Brunauer-Emmett-Teller adsorption analysis), mercury intrusion porosimetry, scanning electron microscopy, or transmission electron microscopy.

In some embodiments, the spherical geometry of the catalyst is a nanosphere. In some embodiments, the catalyst has a diameter of at least 1 nm to at most 500 nm. As used herein, the “diameter” may be the largest linear dimension passing side to side through the center of the nanosphere. In some embodiments, the spherical geometry of the catalyst is a nanosphere having a diameter of at least 1 nm (e.g., at least 5 nm, at least 10 nm, at least 15 nm, at least 20 nm, at least 25 nm, at least 30 nm, at least 35 nm, at least 40 nm, at least 45 nm, at least 50 nm) and/or to at most 500 nm (e.g., at most 450 nm, at most 425 nm, at most 400 nm, at most 375 nm, at most 350 nm, at most 325 nm, at most 300 nm, at most 275 nm, at most 250 nm, at most 225 nm, at most 200 nm, at most 175 nm, at most 150 nm, or at most 100 nm).

In some embodiments, the catalyst includes a mixed metal oxide support. In some embodiments, the mixed metal oxide support includes one or more transition metals, one or more lanthanides, or combinations thereof. In some embodiments, the metal oxide comprises oxygen vacancies, where an oxygen atom is missing from a crystal lattice of the mixed metal oxide support. In some embodiments, the metal oxide comprises a uniform crystal phase where a single crystalline structure is present throughout a portion or all of the mixed metal oxide support. In some embodiments, the uniform crystal phase includes atoms of the mixed metal oxide support that are arranged in a repeating pattern throughout the crystal lattice of the mixed metal oxide support. In some embodiments, the mixed metal oxide support includes titanium oxide, cerium oxide, lanthanum oxide, zirconium oxide, silicon oxide, cobalt oxide, iron oxide, zinc oxide, gallium oxide, germanium oxide, vanadium oxide, manganese oxide, chromium oxide, or combinations thereof. In one particular embodiment, the mixed metal oxide support includes a combination of titanium oxide and cerium oxide. In some embodiments, the mixed metal oxide support has a molar ratio of a first metal to a second metal in the mixed metal oxide (e.g., Ce:Ti in CeTiOx) that ranges from at least 7:3 (e.g., at least 6.5:3.5, at least 6:4, or at least 5.5:4.5) and/or to at most 3:7 (e.g., at most 3.5:6.5, at most 4:6, at most 4.5:5.5, or at most 5:5).

In some embodiments, the catalyst includes a metal coupled to the metal oxide support. Without wishing to be bound to a particular theory, it is contemplated that suitable metals include those that have a combination of active species that promote the production of CO and the chain growth C—C coupling which promote the hydrocarbon production. In some embodiments, the metal includes iron, copper, nickel, palladium, platinum, cobalt, or a combination thereof. In one non-limiting example, it is contemplated that Fe provides a combination of active species (Fe²⁺/Fe³⁺) that may promote the production of CO and the chain growth C—C coupling which may promote the hydrocarbon production, and it is further contemplated that Cu, Ni, Pd, Pt, or Co may be responsible for enhancing production of CO that can be utilized in the Fe phase to enhance the chain growth. In some embodiments, the catalyst includes a first metal (e.g., Cu) and a second metal (Fe) coupled to the metal oxide support. In some embodiments, the catalyst includes a molar ratio of the first metal to the second metal (e.g., Cu:Fe) from at least 1:1 (e.g., at least 1:1.5, or at least 1:2) and/or to at most 1:5 (e.g., at most 1:4.5, at most 1:4, at most 1:3.5, or at most 1:3).

In some embodiments, a total amount of metal coupled to the metal oxide support ranges in an amount from 0.1 wt. % to 50 wt. %, based on a total weight of the catalyst. In some embodiments, the total amount of metal coupled to the metal oxide support is at least 0.1 wt. % (e.g., at least 1 wt. %, at least 2 wt. %, at least 3 wt. %, at least 4 wt. %, at least 5 wt. %, at least 10 wt. %, at least 15 wt. %) and/or to at most 50 wt. % (e.g., at most 45 wt. %, at most 40 wt. %, at most 35 wt. %, at most 30 wt. %, at most 25 wt. %, at most 20 wt. %). In some embodiments, a first metal coupled to the metal oxide support is present in an amount from 0.1 to 25 wt. %, based on the total weight of the catalyst. In some embodiments, the first metal coupled to the metal oxide support is present in an amount from at least 0.1 wt. % (e.g., at least 1 wt. %, at least 2 wt. %, at least 3 wt. %, at least 4 wt. %, at least 5 wt. %, or at least 10 wt. %) and/or to at most 25 wt. %, based on the total weight of the catalyst. In some embodiments, a second metal coupled to the metal oxide support is present in an amount from 0.1 to 25 wt. %, based on the total weight of the catalyst. In some embodiments, the second metal coupled to the metal oxide support is present in an amount from at least 0.1 wt. % (e.g., at least 1 wt. %, at least 2 wt. %, at least 3 wt. %, at least 4 wt. %, at least 5 wt. %, or at least 10 wt. %) and/or to at most 25 wt. %, based on the total weight of the catalyst.

In some embodiments, the catalyst includes a promoter coupled to the mixed metal oxide support. In some embodiments, the promoter includes, but is not limited to, potassium, Na, Cs, Li Mg, Ca, or combinations thereof. In some embodiments, the promoter is present in the catalyst in an amount from 0.1 to 5 wt. %. In some embodiments, the promoter is present in an amount from at least 0.1 wt. % (e.g., at least 0.2 wt. %, at least 0.3 wt. %, at least 0.4 wt. %, at least 0.5 wt. %, at least 1 wt. %, at least 2 wt. %) and/or to at most 5 wt. % (e.g., at most 4 wt. %, or at most 3 wt. %).

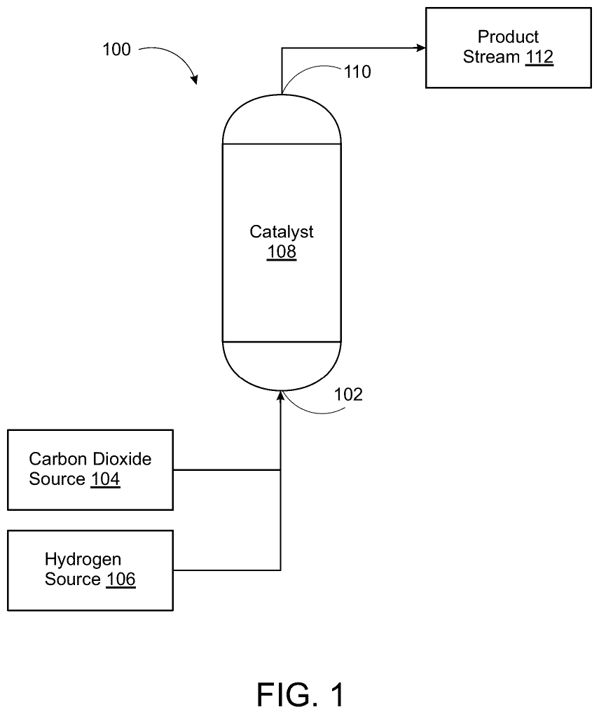

Referring to FIG. 1, a reactor 100 is illustrated according to embodiments of the present disclosure. In some embodiments, the reactor 100 has an inlet 102 in fluid communication with a carbon dioxide source 104 and a hydrogen source 106. The carbon dioxide source 104 includes carbon dioxide and the hydrogen source 106 includes hydrogen. The carbon dioxide and the hydrogen may be transported from the carbon dioxide source 104 and the hydrogen source 106, respectively, in any suitable manner. For example, a compressor (not shown) may be used to transport the carbon dioxide and the hydrogen to the reactor 100. In another example, the carbon dioxide source 102 and/or the hydrogen source 104 may be stored in pressurized tanks that can deliver carbon dioxide and hydrogen, respectively, to the reactor 100 by opening and closing a valve (not shown) positioned between the reactor 100 and either the carbon dioxide source 104 and the hydrogen source 106.

In some embodiments, the reactor 100 includes the catalyst 108 disposed within the reactor 100. The catalyst 108 may be disposed in the reactor 100 in any suitable way such that, when the carbon dioxide and hydrogen enter the reactor 100 through the inlet 102, the hydrogen and carbon dioxide contact the catalyst 108. For example, the reactor 100 may be a fixed bed reactor, a fluidized bed reactor, a trickle bed reactor, or a combination thereof. In some embodiments, when the carbon dioxide and the hydrogen contact the catalyst 108, the catalyst 108 produces a product stream 112 that exits the reactor 100 via an outlet 110. In some embodiments, the product stream 112 includes an unsubstituted straight or branched chained alkane, an unsubstituted straight or branched chained olefin, an unsubstituted straight or branched chained alcohols.

In some embodiments, the unsubstituted straight or branched chained alkane includes C₁₋₃₀ alkanes or C₅₋₁₅ alkanes. In one particular example, the unsubstituted straight or branched chained alkane may include, but is not limited to, pentane and isomers thereof, hexane and isomers thereof, heptane and isomers thereof, octane and isomers thereof, nonane and isomers thereof, decane and isomers thereof, undecane and isomers thereof, dodecane and isomers thereof, tridecane and isomers thereof, tetradecane and isomers thereof, pentadecane and isomers thereof, or combinations thereof.

Suitable isomers of pentane may include, but are not limited to, methylbutane or dimethylpropane. Suitable isomers of hexane may include, but are not limited to, methylpentane or dimethylbutane. Suitable isomers of heptane may include, but are not limited to, methylhexane, dimethylpenane, ethylpentane, or trimethylbutane. Suitable isomers of octane may include, but are not limited to, methylheptane, dimethylhexane, ethylhexane, trimethylpetane, or tetramethylbutane. Suitable isomers of nonane may include, but are not limited to, methyloctane, dimethylheptane, ethylheptane, trimethylhexane, or tetramethylpentane. Suitable isomers of decane may include, but are not limited to, methylnonane, dimethyloctane, ethyloctane, propylheptane, ethylmethylheptane, trimethylheptane, methylpropylhexane, diethylhexane, ethyldimethylhexane, tetramethylhexane, dimethylpropylpentane, diethylmethylpentane, ethyltrimethylpentane, or pentamethylpentane. Suitable isomers of undecane may include, but are not limited to, methyldecane, ethylnonane, dimethylnonane, propyloctane, ethylmethyloctane, trimethyloctane, diethylheptane, ethylmethylheptane, tetramethylheptane, methylpropylheptane, tert-butylheptane, pentamethylhexane, ethyltrimethylhexane, diethylmethylhexane, dimethylpropylhexane, hexamethylpentane, ethyltetramethylpentane, diethyldimethylpentane, or trimethylpropylpentane.

Suitable isomers of dodecane may include, but are not limited to, methylundecane, dimethyldecane, ethyldecane, trimethylnonane, ethylmethylnonane, propylnonane, tetramethyloctane, ethyldimethyloctane, diethyloctane, methylpropyloctane, tert-butyloctane, pentamethylheptane, ethyltrimethylheptane, diethylmethylheptane, dimethylpropyl heptane, ethylpropylheptane, propyldimethylheptane, hexamethylhexane, ethyltetramethylhexane, diethyldimethylhexane, triethylhexane, trimethylpropylhexane, ethylmehtylpropylhexane, tert-butyldimethylhexane, ethylpentamethylpentane, diethyltrimethylpentane, tetramethylpropylpentane, or ethyldimethylpropylpentane. Suitable isomers of tridecane may include, but are not limited to, methyldodecane, dimethylundecane, ethylundecane, trimethyldecane, ethylmethyldecane, propyldecane, tetramethylnonane, ethyldimethylnonane, diethylnonane, methylpropylnonane, butylnonane, pentamethyloctane, ethyltrimethyloctane, diethylmethyloctane, dimethylpropyloctane, ethylpropyloctane, butylmethyloctane, hexamethylheptane, ethyltetramethylheptane, diethyldimethylheptane, triethylheptane, trimethylpropylheptane, ethylmethylpropylheptane, dipropylheptane, dimethylbutylheptane, ethylbutylheptane, ethylpentamethylhexane, diethyltrimethylhexane, triethylmethylhexane, tetramethylpropylhexane, ethyldimethylpropylhexane, methylbis(propyl)hexane, butyltrimethylhexane, diethyltetramethylpentane, pentamethylpropylpentane, ethyltimethylpropylpentane, dimethylbi(propyl)pentane, butyltetramethylpentane)

Suitable isomers of tetradecane may include, but are not limited to, methyltridecane, dimethyldodecane, ethyldodecane, trimethylundecane, ethylmethylundecane, propylundecane, tetramethyldecane, ethyldimethyldecane, diethyldecane, methylpropyldecane, butyldecane, pentamethylnonane, ethyltrimethylnonane, diethylmethylnonane, dimethylpropylnonane, ethylpropylnonane, butylmethylnonane, dimethylethylmethylnonane, dimethylpropylnonane, hexamethyloctane, ethyltatramethyloctane, diethyldimethyloctane, triethyloctane, trimethylpropyloctane, ethylmethylpropyloctane, dipropyloctane, dimethylmethylpropyloctane, ethylbuytloctane, dimethylethylethyloctane, dimethylethyldimethyloctane, heptamethylheptane, ethylpentamethylheptane, ethylpropylmethyloctane, triethylmethylheptane, tetramethylpropylheptane, diethyltrimethylheptane, diethylpropylheptane, methyldipropylheptane, ethyldimethylpropylheptane, butylethylmethylheptane, butyltrimethylheptane, ethylmethylpropylheptane, butylethylmethylheptane, butylpropylheptane, octamethylhexane, ethylhexamethylhexane, diethytetramethylhexane, tiethyldimethylhexane, tetraethylhexane, pentamethylpropylhexane, ethyltrimethylpropylhexane, diethylmethylpropylhexane, dimethyldipropylhexane, tertbutyltetramethylhexane, ethyltetramethyl(methylethyl)pentane, trimethylbis(methylethyl)pentane, or dimethylethylpentamethylpentane.

Suitable isomers of pentadecane may include, but are not limited to, methyltetradecane, dimethyltridecane, ethyltridecane, trimethyldodecane, ethylmethyltridecane, propyltridecane, tetramethylundecane, ethyldimethylundecane, diethylundecane, methylpropylundecane, butylundecane, pentamethyldecane, ethyltrimethyldecane, diethylmethyldecane, dimethylpropyldecane, ethylpropyldecane, butylmethyldecane, dimethylethylmethyldecane, dimethylpropyldecane, ethylpropyldecane, hexamethylnonane, ethyltetramethylnonane, diethyldimethylnonane, triethylnonane, trimethylpropylnonane, ethylmethylpropylnonane, dipropylnonane, dimethylmethylpropylnonane, dimethylethyldimethylnonane, ethylbutylnonane, dimethylethylethylnonane, ethylpropylmethylnonane, heptamethyloctane, ethylpentamethyloctane, diethyltrimethyloctane, triethylmethyloctane, tetramethylpropyloctane, ethyldimethylpropyloctane, diethylpropyloctane, methyldipropyloctane, butyltrimethyloctane, ethylmethylpropyloctane, butylethylmethyloctane, butylpropyloctane, octamethylheptane, ethylhexamethylheptane, diethytetramethylheptane, tiethyldimethylheptane, tetraethylheptane, pentamethylpropylheptane, ethyltrimethylpropylheptane, diethylmethylpropylheptane, dimethyldipropylheptane, tertbutyltetramethylheptane, ethyltetramethyl(methylethyl)hexane, trimethylbis(methylethyl)hexane, or dimethylethylpentamethylhexane.

In some embodiments, the catalyst 108 hydrogenates CO₂ to produce a product stream 112 with an unsubstituted straight or branched chained C₅₋₁₅ alkane selectivity from 5% to 70%. In some embodiments, the catalyst 108 hydrogenates CO₂ to produce the product stream 112 with an unsubstituted straight or branched chained C₅₋₁₅ selectivity of at least 5% (e.g., at least 10%, at least 15%, at least 20%, at least 25%, at least 30%, or at least 35%) and/or to at most 70% (e.g., at most 60%, at most 50%, or at most 40%). As used herein, “C₅₋₁₅ alkane selectivity” expressed as a percentage may refer to (moles of CO₂ converted to C₅₋₁₅ alkanes)/(moles of CO₂ consumed)*(100)−(selectivity, %).

In some embodiments, the product stream 112 includes an unsubstituted straight or branched chained C₂₊ olefin, which may include, but is not limited to, C₂₋₅ olefins. In some embodiments, the C₂₋₅ olefins include, but are not limited to, ethylene, propylene, 1-butene, 2-butene, isobutylene, 1-pentene, 2-pentene, 2-methyl-1-butene, 3-methyl-1-butene, 2-methyl-2-butene, or combinations thereof.

In some embodiments, the catalyst 108 hydrogenates CO₂ to produce a product stream 112 with an unsubstituted straight or branched chained C₂₋₅ olefins selectivity from 5% to 45%. In some embodiments, the catalyst 108 hydrogenates CO₂ to produce the product stream 112 with an unsubstituted straight or branched chained C₂₋₅ olefins selectivity of at least 5% (e.g., at least 10%, at least 15%, or at least 20%) and/or to at most 45% (e.g., at most 40%, at most 35%, at most 30%, or at most 25%). As used herein, “C₂₋₅ olefin selectivity” expressed as a percentage may refer to (moles of CO₂ converted to C₂₋₅ olefin)/(moles of CO₂ consumed)*(100)−(selectivity, %). In some embodiments, the product stream 112 includes an unsubstituted straight or branched C₂₊ alcohol, which may include, but is not limited to, C₂₋₅ alcohols. In some embodiments, unsubstituted straight or branched C₂₋₅ alcohols include, but are not limited to, ethanol, 1-propanol, 2-propanol, 1-butanol, 2-butanol, isobutanol, tert-butyl alcohol, 1-pentanol, 2-pentanol, 3-pentanol, 2-methyl-1-butanol, 3-methyl-1-butanol, 3-methyl-1-butanol, 2-methyl-2-butanol, 2-methyl-3-butanol, 2,2-dimethylpropanol, or combinations thereof.

In some embodiments, the catalyst 108 hydrogenates CO₂ to produce a product stream 112 with an unsubstituted straight or branched chained C₂₋₅ alcohol selectivity from 0.1% to 20%. In some embodiments, the catalyst 108 hydrogenates CO₂ to produce the product stream 112 with an unsubstituted straight or branched chained C₂₋₅ alcohol selectivity of at least 0.1% (e.g., at least 1%, at least 5%, or at least 10%) and/or to at most 20% (e.g., at most 19%, at most 18%, at most 17%, or at most 16%, or at most 15%). As used herein, “C₂₋₅ alcohol selectivity” expressed as a percentage may refer to (moles of CO₂ converted to C₂₋₅ alcohol)/(moles of CO₂ consumed)*(100)=(selectivity, %).

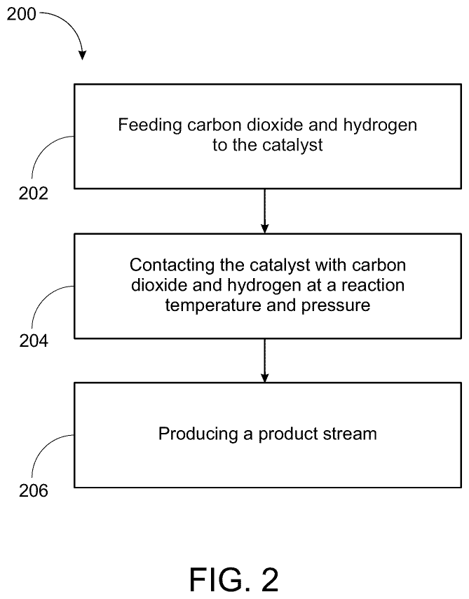

Referring to FIG. 2, a method 200 is provided according to embodiments of the present disclosure. In some embodiments, at operation 202 the method 200 includes feeding carbon dioxide and hydrogen to the catalyst 108. For example, operation 202 may include feeding the carbon dioxide from a carbon dioxide source 104 and feeding the hydrogen from a hydrogen source 106 to the catalyst 108. In some embodiments, the catalyst 108 is positioned in a reactor 100. In one example, a compressor may be used to transport the carbon dioxide and the hydrogen to the catalyst 108. In another example, the carbon dioxide source 102 and/or the hydrogen source 104 may be stored in pressurized tanks that can deliver carbon dioxide and hydrogen, respectively, to the reactor 100 by opening and closing a valve positioned between the reactor 100 and either the carbon dioxide source 104 and the hydrogen source 106.

At operation 204, the method 200 includes contacting the catalyst 108 with carbon dioxide and hydrogen at a reaction temperature and pressure. In some embodiments, operation 204 includes catalyst with the carbon dioxide and the hydrogen occurs at a gas hourly space velocity (GHSV) from 1,500 to 12,000 l/h. In some embodiments, the GHSV is at least 1,500 (e.g., at least 2,000, at least 2,500, at least 3,000, at least 3,500, at least 4,000, at least 4,500, at least 5,000, at least 5,500, or at least 6,000 l/h) and/or to at most 12,000 l/h (e.g., at most 11,000, at most 10,000, at most 9,000, at most 8,000, or at most 7,000 l/h).

In some embodiments, operation 204 includes contacting the catalyst 108 with the carbon dioxide and the hydrogen at a temperature from 250° C. to 400° C. In some embodiments, the temperature is at least 250° C. (e.g., at least 275, at least 300, or at least 325° C.) and/or at most 400° C. (e.g., at most 375° C. or at most 350° C.). In some embodiments, operation 204 includes contacting the catalyst 108 with the carbon dioxide and the hydrogen at a pressure from 20 bar to 100 bar. In some embodiments, the pressure is at least 20 bar (e.g., at least 30 bar, at least 40 bar, or at least 50 bar) and/or to at most 100 bar (e.g., at most 90 bar, at most 80 bar, at most 70 bar, or at most 60 bar). At operation 206, the method 200 includes producing the product stream 112, which may exit the reactor 100 via the outlet 110.

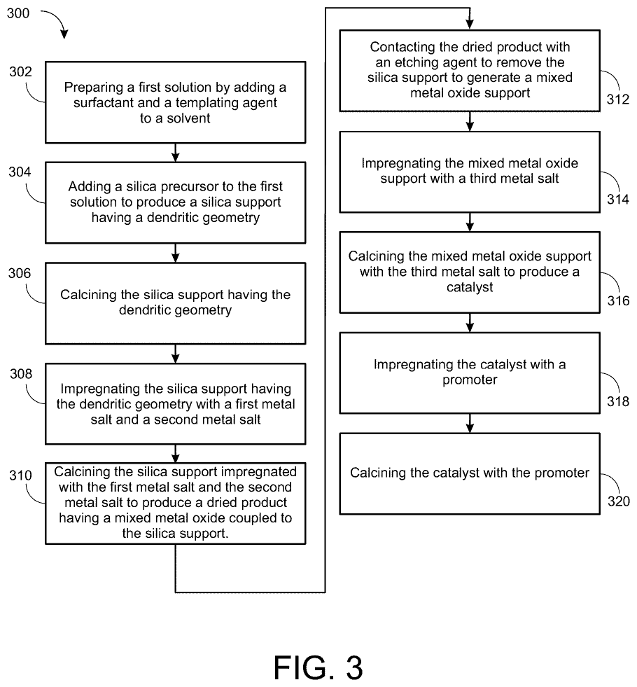

Referring to FIG. 3, the present disclosure provides a method 300 of synthesizing the catalyst 108. At operation 302, the method 300 includes preparing a first solution. In some embodiments, operation 302 includes adding a surfactant and a templating agent into a solvent to prepare the first solution. In some embodiments, the surfactant is an amino surfactant. Suitable amino surfactants include, but is not limited to, triethanolamine (TEA). In some embodiments, the solvent is water, such as deionized water. In some embodiments, operation 302 includes heating the first solution to a temperature for a duration. In some embodiments, the temperature is from 50° C. to 90° C., and the duration in operation 302 is at least 5 minutes to an hour. Operation 302 further includes adding a templating agent to the first solution and stirring the first solution for a duration. In some embodiments, the templating agent may include cetyltrimethylammonium bromide (CTAB), sodium salicylate (NaSal), or combinations thereof. In some embodiments, the duration of adding the templating agent in operation 302 is at least 5 minutes to two hours.

At operation 304, the method 300 includes adding a silica precursor to the first solution to produce a silica support having a dendritic geometry. In some embodiments, the silica support having the dendritic geometry is a dendritic mesoporous silica nanoparticle (DMSNs). In some embodiments, the silica precursor includes, but is not limited to, tetraethyl orthosilicate (TEOS). In some embodiments, operation 304 includes adding the silica precursor intermittently over a duration at a reaction temperature (e.g., the duration may be at least 30 minutes to three hours and the temperature may range from 50° C. to 90° C.). In some embodiments, operation 304 further includes heating the first solution at a temperature for a duration to evaporate the solvent (e.g., at least 100° C. for two to five hours) to obtain a dried product that includes the silica support having the dendritic geometry. In some embodiments, operation 304 further includes purifying the silica support, which may include filtering and desiccation.

At operation 306, the method 300 includes calcining the silica support having the dendritic geometry. In some embodiments, calcining the silica support having the dendritic geometry includes heating the silica support having the dendritic geometry to a temperature from at least 550° C. to 800° C. In some embodiments, heating to the temperature may occur with a temperature ramp (e.g., 5 hours from ambient temperature to a temperature at least 550° C. to 800° C.) over a period of time (e.g., from two to ten hours, or from three to six hours).

At operation 308, the method 300 includes impregnating the silica support having the dendritic geometry with a first metal salt and a second metal salt. For example, operation 308 may include adding a first metal salt and a second metal salt to a second solvent to form a second solution. In some embodiments, the salts are added in a molar ratio of a first metal salt to a second metal salt that ranges from 7:3 to 3:7. In some embodiments, the first metal salt is titanium-based metal salt such as titanium chloride (e.g., TiCl₄). In some embodiments the second metal salt is a cerium-based metal salt, such as cerium nitrate (e.g., Ce(NO₃)₃). In some embodiments, the second solution is an organic solvent, such as acetone. Operation 308 may include mixing the second solution until it become clear. In some embodiments, operation 308 further includes adding the silica support having the dendritic geometry to the second solution for a duration (e.g., at least two hours). Operation 308 may further include heating the second solution to evaporate the second solvent to obtain a dried product comprising the silica support having the dendritic geometry impregnated with the first metal and the second metal.

At operation 310, the method 300 includes calcining the dried product comprising the silica support impregnated with the first metal and the second metal. In some embodiments, operation 310 includes a first calcination at a temperature of at least 200° C. to 400° C. and for a duration from at least two to eight hours. In some embodiments, following the first calcination, operation 308 is repeated using a reduced amount of metal salts (e.g., in an amount of ⅔ relative to the first performance of operations 308). In some embodiments, operation 310 includes calcining the silica support with the subsequent impregnation of the first metal salt and the second metal salt a second time to produce a dried product comprising a mixed metal oxide coupled to the silica support having the dendritic geometry. In some embodiments, the second calcination occurs at a temperature of at least 500° C. to 800° C. over a duration of at least two hours to five hours. In some embodiments, operation 310 includes calcining at a temperature ramp of 1° C. min⁻¹ to decompose any inorganic precursors.

At operation 312, the method 300 includes contacting the dried product comprising the mixed metal oxide coupled to the silica support having the dendritic geometry with an etching agent. In some embodiments, the etching agent removes the silica support to generate a mixed metal oxide having the dendritic geometry. In some embodiments, the etching agent is sodium hydroxide. In one particular example, contacting the dried product comprising the mixed metal oxide coupled to the silica support may include contacting the dried product with a 2M solution of NaOH. In some embodiments, the solution is heated to a temperature between room temperature and 70° C. while stirring (e.g., for at least 1 hr). In some embodiments, operation 312 is repeated multiple times (e.g., repeated at least one time, at least two times, to at most three times, or at most five times). In one particular example, the mixed metal oxide having the dendritic geometry is a CeₓTi₁₋ₓO₂ composite.

At operation 314, the method 300 includes impregnating the mixed metal oxide having the dendritic geometry with a third metal salt. For example, operation 314 may include contacting the mixed metal oxide with a third metal salt in solution, such as acetone, to generate an impregnated mixed metal oxide. In some embodiments, the metal salt includes, but is not limited to, iron (III) nitrate, copper nitrate, or combinations thereof. In some embodiments, operation 314 further includes drying the impregnated mixed metal oxide at a temperature from ambient temperature to 100° C.

At operation 316, the method 300 includes calcining the mixed metal oxide support with the third metal salt to produce a catalyst 108. In some embodiments, the catalyst 108 comprises the mixed metal oxide support having the dendritic geometry and the metal coupled to the metal oxide support. In some embodiments, operation 316 includes calcining at a temperature of at least 400° C. to 800° C. for at least two to six hours to obtain the catalyst 108.

At operation 318, the method 300 includes impregnating the catalyst 108 with a promoter. For example, operation 318 may include contacting the catalyst 108 a promoter in a solution. In some embodiments, operation 318 includes contacting the catalyst 108 with potassium nitrate dissolved in acetone. Operation 318 may further include drying the catalyst 108 impregnated with the promoter at a temperature from ambient temperature to 100° C.

At operation 320, the method 300 includes calcining the catalyst 108 impregnated with the promoter. In some embodiments, operation 320 includes calcining at a temperature from at least 400° C. to 800° C. for at least two to six hours to obtain the catalyst 108 comprising the promoter coupled to the mixed metal oxide support.

In an example implementation, a composition is provided. The composition includes a catalyst that includes a mixed metal oxide support having a dendritic geometry and a metal coupled the metal oxide support. In the example implementation, when the catalyst contacts carbon dioxide and hydrogen, the catalyst produces a product stream comprising an unsubstituted straight or branched chained C₅₋₁₅ alkane.

In an example implementation combinable with any other example implementation, where the product stream further comprises an unsubstituted straight or branched chained C₂-C₅ olefins, an unsubstituted straight or branched chained C₂-C₅ alcohols, or a combination thereof.

In an example implementation combinable with any other example implementation, where the catalyst comprises a pore diameter from 2 nm to 50 nm. In some implementations, the catalyst comprises a pore diameter from 2 nm to 10 nm.

In some implementations combinable with any other example implementation, where the catalyst has a surface area from 40 to 200 m²/g.

In some implementations combinable with any other example implementation, where the catalyst has a pore volume from 0.2 to 0.7 cc/g

In an example implementation combinable with any other example implementation, where the mixed metal oxide support comprises titanium oxide and cerium oxide. In some implementations, the mixed metal oxide support has a molar ratio of Ce:Ti from 7:3 to 3:7.

In an example implementation combinable with any other example implementation, where the metal is present in the catalyst in an amount from 0.1 to 40 wt. %, based on a total weight of the catalyst.

In an example implementation combinable with any other example implementation, where the metal comprises iron, copper, or a combination thereof. In some implementations, the metal has a molar ratio of Cu:Fe from 1:1 to 5:1.

In an example implementation combinable with any other example implementation, where the dendritic geometry comprises a central core having a plurality of arms that extend from the central core and branch at a plurality of branching points to form a spherical geometry, wherein the dendritic geometry comprises a plurality of interconnected pores dispersed throughout the dendritic geometry.

In an example implementation combinable with any other example implementation, where the spherical geometry is a nanosphere having a diameter from 5 nm to 500 nm.

In an example implementation combinable with any other example implementation, where the composition further includes a promoter coupled to the mixed metal oxide support.

In an example implementation combinable with any other example implementation, where the mixed metal oxide support comprises titanium oxide and cerium oxide; where the mixed metal oxide support has a molar ratio of Ce:Ti from 7:3 to 3:7; where the metal comprises iron and copper, and the metal has a molar ratio of Cu:Fe from 1:1 to 5:1.

In an example implementation combinable with any other example implementation, where a reactor is provided. The reactor includes an inlet in fluid communication with a carbon dioxide source and a hydrogen source, the carbon dioxide source comprising carbon dioxide, and the hydrogen source comprising hydrogen. The catalyst of any other example implementation is disposed within the reactor.

In an example implementation, a method is provided. The method includes contacting a catalyst with carbon dioxide and hydrogen to produce a product stream comprising an unsubstituted straight or branched chained C₅₋₁₅ alkane. In some implementations, the catalyst includes a mixed metal oxide support having a dendritic geometry and a metal coupled the metal oxide support.

In an example implementation combinable with any other example implementation, where contacting the catalyst with the carbon dioxide and the hydrogen occurs at a temperature from 250° C. to 400° C.

In an example implementation combinable with any other example implementation, where contacting the catalyst with the carbon dioxide and the hydrogen occurs at a pressure from 20 bar to 100 bar.

In an example implementation combinable with any other example implementation, where contacting the catalyst with the carbon dioxide and the hydrogen occurs at a gas hourly space velocity from 1,500 to 12,000.

In an example implementation combinable with any other example implementation, where during contacting the catalyst with the carbon dioxide and the hydrogen, the mixed metal oxide comprises titanium oxide and cerium oxide.

In an example implementation combinable with any other example implementation, where during contacting the catalyst with the carbon dioxide and the hydrogen, the metal comprises iron, copper, or a combination thereof.

In an example implementation combinable with any other example implementation, where during contacting the catalyst with the carbon dioxide and the hydrogen, the catalyst further comprises a promoter coupled to the mixed metal oxide.

In an example implementation combinable with any other example implementation, where during contacting the catalyst with the carbon dioxide and the hydrogen, the catalyst comprises a central core having a plurality of arms that extend from the central core and branch at a plurality of branching points to form a spherical geometry, wherein the dendritic geometry comprises a plurality of interconnected pores dispersed throughout the dendritic geometry.

In an example implementation combinable with any other example implementation, where during contacting the catalyst with the carbon dioxide and the hydrogen: the mixed metal oxide comprises titanium oxide and cerium oxide; the metal comprises iron, copper, or a combination thereof; the catalyst comprises a central core having a plurality of branching arms that extend from the central core to form a nanosphere having a diameter from 5 nm to 500 nm; and the catalyst further comprises a promoter coupled to the mixed metal oxide support, wherein the promoter comprises potassium.

In an example implementation, a method is provided. The method includes adding a surfactant and a templating agent to a first solvent to produce a first solution and adding a silica precursor to the first solution to produce a silica support having a dendritic geometry. The method includes calcining the silica support having the dendritic geometry and impregnating the silica support having the dendritic geometry with a first metal salt and a second metal salt. The method includes calcining the silica support having the dendritic geometry with the first metal salt and the second metal salt to produce a dried product comprising a mixed metal oxide coupled to the silica support having the dendritic geometry and contacting the dried powder with an etching agent, wherein the etching agent removes the silica support to generate a mixed metal oxide support having the dendritic geometry.

In an example implementation combinable with any other example implementation, where the method further includes impregnating the mixed metal oxide support having the dendritic geometry with a third metal salt and calcining the mixed metal oxide support with the third metal salt to produce a catalyst comprising the mixed metal oxide support having the dendritic geometry and the third metal coupled to the metal oxide support.

In an example implementation combinable with any other example implementation, where the method further includes impregnating the catalyst with a promoter and calcining the catalyst with the promoter to produce a catalyst comprising the promoter coupled to the mixed metal oxide support.

In an example implementation combinable with any other example implementation, where impregnating the silica support includes impregnating the silica support with the first metal salt that comprises a titanium-based salt and impregnating the silica support with the second metal salt comprises a cerium-based salt.

In an example implementation combinable with any other example implementation, where impregnating the mixed metal oxide support with the third metal salt includes impregnating the mixed metal oxide with the third metal salt that is selected from an iron-based salt, a copper-based salt, or a combination thereof.

### EXAMPLES

### Example 1

### Preparation of Hard Template Dendritic Mesoporous Silica Nanospheres

To begin with, 0.82 g of triethanolamine (TEA) was dissolved in 300 ml of deionized water and stirred at a temperature of 80° C. for a duration of 30 minutes. Following this, 4.56 g of cetyltrimethylammonium bromide (CTAB) and 2.02 g of sodium salicylate (NaSal) were introduced into the solution and the mixture was stirred for an additional hour. Subsequently, 48.0 g of TEOS (28.4 w % SiO2) was gradually added to the solution and stirred continuously for 2.0 hours. The mixture was then transferred to a 500 ml autoclave and heated at 100° C. for 4 hours. Lastly, pure DMSNs were produced through a process of filtration, desiccation, and calcination at 550° C. (5 hours from AT to 550) over a period of 6 hours.

### Dendritic Metal Oxide Composites

Dendritic TiₓCe₁₋ₓO₂ composites were synthesized. In the preparation process, a total 18 mmol of TiCl₄—99.9% and Ce(NO₃)₃, following molar ratio of Ce:Ti, (1:0, 7:3, 5:5, 3:7, 0:1) in sequence was dissolved into 15 mL of acetone. After the solution became clear, 2 g of DMSNs hard template was added and the mixture was stirred for 2 hours in a 100 ml beaker, then the mixture was transferred to a clean flat Petri dish and the solvent was evaporated. In order to achieve higher loadings, the above dried hybrid powder was calcined at 200° C. for 6 h to decompose the metal precursors, and then the impregnation step was repeated, but the amount of precursors was reduced to ⅔ compared to the first step. The resulting samples were calcined in air at 500° C. for 4 h with a heating ramp of 1° C. min⁻¹ to completely decompose the inorganic precursors. Finally, the silica template was removed using a 2 M NaOH (50 mL) aqueous solution at 70° C. to room temperature (stirring for 1 h) and this etching process was repeated three times. The obtained CeₓTi₁₋ₓO₂ composites with different initial Ce/Ti molar ratios (1:0, 7:3, 5:5, 3:7, 0:1) were prepared.

### Dendritic Metal/Metal Oxide Catalyst

Dendritic CeTiOx composites supported Fe—Cu bimetallic catalysts were prepared by co-impregnation method using acetone solution of Fe(III) nitrate nona hydrate and Cu(NO₃)₂·2.5H₂O). The impregnated samples were dried at ambient temperature and calcined in an electric furnace at 723 K for 5 h. Fe and Cu metal loadings were 15 wt. % and 20 wt. %, respectively.

### Promoted Metal/Metal Oxide Catalyst

Dendritic FeCu/CeTiOx composites catalysts were prepared by wet-impregnation method using acetone solution of potassium nitrate. The impregnated samples were dried at ambient temperature and calcined in an electric furnace at 723 K for 4 h. K were kept at 0.4% to 4%.

### CO₂ Hydrogenation

A gas mixture including carbon dioxide and hydrogen were fed to a reactor at a temperature of 350° C., a pressure of 50 bar, and at a gas hourly space velocity (GHSV) of 3,300. In each respective reaction, the reactor had a catalyst according to one of the following samples described in Table 1 below:

| | | Surface | Pore | Pore |
|---|---|---|---|---|
| | | Area | Volume | Diameter |
| Catalyst Composition | Sample | (m²/g) | (cc/g) | (nm) |
| Ce—TiOₓ (1:1) | DCT-1 | 90.8 | 0.5 | 3.2 |
| Ce—TiOₓ (0.3:0.7) | DCT-2 | 68.2 | 0.3 | 3.8 |
| Ce—TiOₓ (0.7:0.3) | DCT-3 | 95.8 | 0.4 | 3.4 |
| CuFe(2:1)/Ce—TiOₓ (1:1) | FC/DCT-1 | 98.8 | 0.4 | 6.6 |
| CuFe(2:1)/Ce—TiOₓ | FC/DCT-2 | 93.3 | 0.4 | 6.5 |
| (0.3:0.7) | | | | |
| CuFe(2:1)/Ce—TiOₓ | FC/DCT-3 | 91.4 | 0.4 | 6.5 |
| (0.7:0.3) | | | | |
| K—CuFe(2:1)/Ce—TiOₓ | K/FC/DCT-1 | 86.3 | 0.3 | 6.6 |
| (1:1) | | | | |
| K—CuFe(2:1)/Ce—TiOₓ | K/FC/DCT-2 | 82.6 | 0.3 | 6.5 |
| (0.3:0.7) | | | | |
| K—CuFe(2:1)/Ce—TiOₓ | K/FC/DCT-3 | 93.2 | 0.4 | 6.6 |
| (0.7:0.3) | | | | |

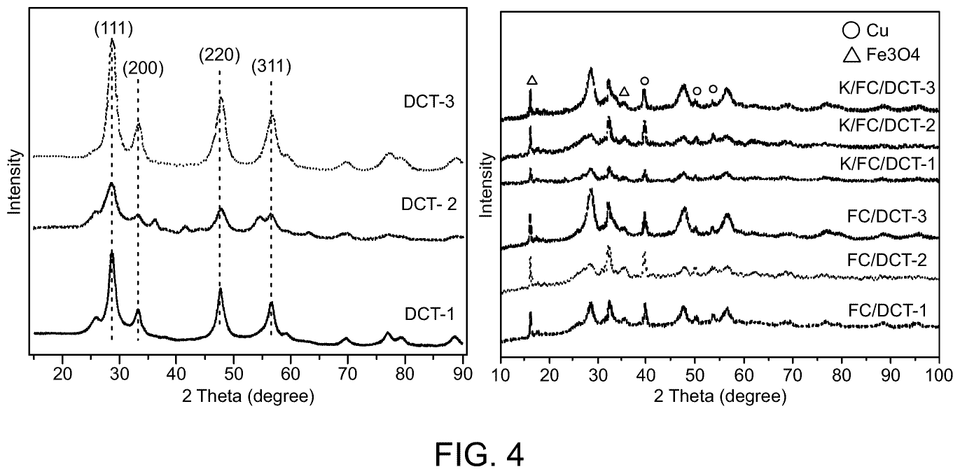

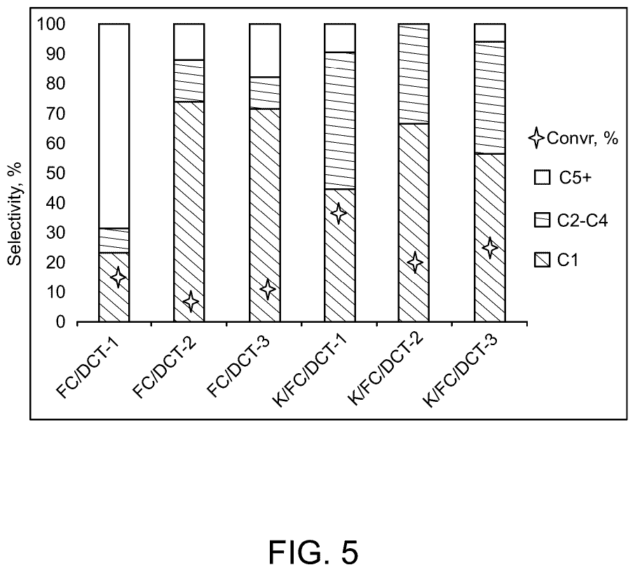

FIG. 4 shows XRD patterns of each respective catalyst. FIG. 5 shows the selectivity for C₅₊ products, C₂₋₄ products, and C₁ products, as well as the overall conversion at a temperature of 350° C., a pressure of 50 bar, and at a gas hourly space velocity (GHSV) of 3,300. The FC/DCT-1 catalyst had a conversion of 15%, a selectivity of C₁ products of 22%, a selectivity of C₂₋₄ products of 10%, and a selectivity of C₅₊ products of 68%. The FC/DCT-2 catalyst had a conversion of 7%, a selectivity of C₁ products of 74%, a selectivity of C₂₋₄ products of 14%, and a selectivity of C₅₊ products of 13%. The FC/DCT-3 catalyst had a conversion of 10%, a selectivity of C₁ products of 71%, a selectivity of C₂₋₄ products of 8%, and a selectivity of C₅₊ products of 21%.

The K/FC/DCT-1 catalyst had a conversion of 38%, a selectivity of C₁ products of 43%, a selectivity of C₂₋₄ products of 50%, and a selectivity of C₅₊ products of 7%. The K/FC/DCT-2 catalyst had a conversion of 20%, a selectivity of C₁ products of 66%, a selectivity of C₂₋₄ products of 34%, and a selectivity of C₅₊ products of 0%. The K/FC/DCT-3 catalyst had a conversion of 25%, a selectivity of C₁ products of 53%, a selectivity of C₂₋₄ products of 43%, and a selectivity of C₅₊ products of 4%.

What is claimed is:

1. A composition comprising:
a catalyst comprising:
a mixed metal oxide support having a dendritic geometry; and
a metal coupled the metal oxide support;
wherein, when the catalyst contacts carbon dioxide and hydrogen, the catalyst produces a product stream comprising an unsubstituted straight or branched chained C₅₋₁₅ alkane.

2. The catalyst of claim 1, wherein the product stream further comprises an unsubstituted straight or branched chained C₂-C₅ olefins, an unsubstituted straight or branched chained C₂-C₅ alcohols, or a combination thereof.

3. The catalyst of claim 1, wherein the catalyst comprises a pore diameter from 2 nm to 50 nm.

4. The catalyst of claim 1, wherein the catalyst comprises a pore diameter from 2 nm to 10 nm.

5. The catalyst of claim 1, wherein the catalyst has a surface area from 40 to 200 m²/g.

6. The catalyst of claim 1, wherein the catalyst has a pore volume from 0.2 to 0.7 cc/g.

7. The catalyst of claim 1, wherein the mixed metal oxide support comprises titanium oxide and cerium oxide.

8. The catalyst of claim 7, wherein the mixed metal oxide support has a molar ratio of Ce:Ti from 7:3 to 3:7.

9. The catalyst of claim 1, wherein the metal is present in the catalyst in an amount from 0.1 to 40 wt. %, based on a total weight of the catalyst.

10. The catalyst of claim 1, wherein the metal comprises iron, copper, or a combination thereof.

11. The catalyst of claim 10, wherein the metal has a molar ratio of Cu:Fe from 1:1 to 5:1.

12. The catalyst of claim 1, wherein the dendritic geometry comprises a central core having a plurality of arms that extend from the central core and branch at a plurality of branching points to form a spherical geometry, wherein the dendritic geometry comprises a plurality of interconnected pores dispersed throughout the dendritic geometry.

13. The catalyst of claim 12, wherein the spherical geometry is a nanosphere having a diameter from 5 nm to 500 nm.

14. The catalyst of claim 1 further comprising a promoter coupled to the mixed metal oxide support.

15. The catalyst of claim 1, wherein the mixed metal oxide support comprises titanium oxide and cerium oxide;
wherein the mixed metal oxide support has a molar ratio of Ce:Ti from 7:3 to 3:7;
wherein the metal comprises iron and copper; and
the metal has a molar ratio of Cu:Fe from 1:1 to 5:1.

16. A reactor comprising:
an inlet in fluid communication with a carbon dioxide source and a hydrogen source, the carbon dioxide source comprising carbon dioxide, and the hydrogen source comprising hydrogen; and
the catalyst of claim 1 disposed within the reactor.

17. A method comprising:
contacting a catalyst with carbon dioxide and hydrogen to produce a product stream comprising an unsubstituted straight or branched chained C₅₋₁₅ alkane,
wherein the catalyst comprises:
a mixed metal oxide support having a dendritic geometry; and
a metal coupled the metal oxide support.

18. The method of claim 17, wherein contacting the catalyst with the carbon dioxide and the hydrogen occurs at a temperature from 250° C. to 400° C.

19. The method of claim 17, wherein contacting the catalyst with the carbon dioxide and the hydrogen occurs at a pressure from 20 bar to 100 bar.

20. A method comprising:
adding a surfactant and a templating agent to a first solvent to produce a first solution;
adding a silica precursor to the first solution to produce a silica support having a dendritic geometry;
calcining the silica support having the dendritic geometry;
impregnating the silica support having the dendritic geometry with a first metal salt and a second metal salt;
calcining the silica support having the dendritic geometry with the first metal salt and the second metal salt to produce a dried product comprising a mixed metal oxide coupled to the silica support having the dendritic geometry; and
contacting the dried powder with an etching agent, wherein the etching agent removes the silica support to generate a mixed metal oxide support having the dendritic geometry.
